(19) United States
(12) Patent Application Publication
(10) Pub. No.: US 2018/0338996 A1
(43) Pub. Date: Nov. 29, 2018

(54) COMBINATION THERAPY FOR TRAUMATIC BRAIN INJURY

(71) Applicants: SUBHRA MOHAPATRA, LUTZ, FL (US); SHYAM S. MOHAPATRA, LUTZ, FL (US); MAHASWETA DAS, TAMPA, FL (US)

(72) Inventors: SUBHRA MOHAPATRA, LUTZ, FL (US); SHYAM S. MOHAPATRA, LUTZ, FL (US); MAHASWETA DAS, TAMPA, FL (US)

(73) Assignee: UNIVERSITY OF SOUTH FLORIDA, TAMPA, FL (US)

(21) Appl. No.: 15/987,580

(22) Filed: May 23, 2018

Related U.S. Application Data
(60) Provisional application No. 62/510,185, filed on May 23, 2017.

(51) Int. Cl. A61K 35/28; A61K 31/4439; A61P 25/00

(52) U.S. Cl. CPC A61K 35/28; A61P 25/00; A61K 31/4439

(57) ABSTRACT

The present invention concerns a method for treatment of traumatic brain injury (TBI) in a human or non-human animal subject, comprising administering stem or progenitor cells to the subject, such as mesenchymal stromal cells; and administering one or more PPARγ agonists, such as pioglitazone (PG), to the subject before, during, and/or after administration of the stem or progenitor cells. Another aspect of the invention concerns a pharmaceutical composition useful for treating TBI, the composition comprising stem cells or progenitor cells, such as mesenchymal stromal cells, and one or more PPARγ agonists, such as PG.

### CROSS-REFERENCE TO RELATED APPLICATION

The present application claims the benefit of U.S. Provisional Application Ser. No. 62/510,185, filed May 23, 2017, which is hereby incorporated by reference herein in its entirety, including any figures, tables, nucleic acid sequences, amino acid sequences, or drawings.

### GOVERNMENT SUPPORT

This invention was made with government support under Grant No. BX002668 awarded by the VA Merit Review. The government has certain rights in the invention.

### BACKGROUND OF THE INVENTION

Inflammation in the brain plays a key role in neurodegeneration and subsequent disabilities following traumatic brain injury (TBI). Although stem cell therapy (SCT) is under investigation as a promising therapeutic approach for TBI, its clinical use has been limited.

### BRIEF SUMMARY OF THE INVENTION

C-C motif chemokine ligand 20 (CCL20) plays an important role in mediating secondary neurodegeneration following mild TBI (Das et al., 2011), and peroxisome proliferator-activated receptor gamma (PPARγ) agonist pioglitazone (PG) reduces CCL20 expression in the brain following TBI. The inventors tested the hypothesis that treatment with a PPARγ agonist, such as PG, would improve the outcome of SCT following TBI using a lateral fluid percussion injury (LFPI) model in rats. Neurodegeneration and CC120 expression were analysed in the brain by immunohistochemical method by using specific antibodies. Quantitation was performed using image J program. Combined PG and human mesenchymal stromal cell (hMSC) treatment significantly reduced the number of degenerated neurons 7 days after TBI. Although PG or hMSC treatment reduces CCL20 expression in the cortex after TBI, the effect is more pronounced after combined pioglitazone and hMSC treatment. The results in these studies demonstrate that improved SCT outcome following TBI may be achieved by a combination therapy comprising administration of a PPARγ agonist, such as PG, with stem or progenitor cell treatment.

One aspect of the invention concerns a method for treatment of traumatic brain injury (TBI) in a human or non-human animal subject, comprising administering stem cells or progenitor cells to the subject, such as mesenchymal stromal cells or mesenchymal progenitor cells; and administering one or more PPARγ agonists, such as PG, to the subject before, during, and/or after administration of the stem cells or progenitor cells.

Another aspect of the invention concerns a pharmaceutical composition that may be administered for the treatment of TBI, the composition comprising stem cells or progenitor cells, such as mesenchymal stromal cells or mesenchymal progenitor cells, and one or more PPARγ agonists, such as PG.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIGS. 1A-1B. Biodistribution of hMSCS in rat after TBI. FIG. 1A shows ex vivo IVIS imaging of different organs showing the DiR fluorescence. 1 million DiR-labeled hMSCs were administered 3 days post TBI. Rats were euthanized 8 days post-TBI, organs were harvested and imaged. Naíve animal was used as control. FIG. 1B shows quantitation of DiR fluorescence in different organs.

FIGS. 2A-2B. Combined Pioglitazone and hMSC treatment significantly reduces neuronal degeneration in the cortex of rat 7 days post TBI. FIG. 2A shows fluorescence microscopic images showing fluorojade staining in the cortex showing the degenerating neurons. FIG. 2B shows the average FJ positive neurons after different treatments 7 days post TBI.

FIGS. 3A-3B. Combined Pioglitazone and hMSC treatment reduces CCL20 expression in the rat cortex 7 days post TBI. FIG. 3A shows bright field images with immunostaining of CCL20 under different treatments. FIG. 3B shows CCL20 immunoreactivity (Mean±SEM) as quantitated by Image J.

### DETAILED DESCRIPTION OF THE INVENTION

One aspect of the invention concerns a method for treatment of traumatic brain injury (TBI) in a human or non-human animal subject, comprising administering stem cells or progenitor cells to the subject, such as mesenchymal stromal cells; and administering one or more PPARγ agonists, such as pioglitazone (PG), to the subject before, during, and/or after administration of the stem or progenitor cells. In some embodiments, the one or more PPARγ agonists are administered prior to administration of the cells.

Another aspect of the invention concerns a pharmaceutical composition useful for treating TBI, comprising stem or progenitor cells, such as mesenchymal stromal cells, and one or more PPARγ agonists, such as PG, which may be administered to a subject according to the methods of the invention. The compositions may further comprise a pharmaceutically acceptable carrier or diluent. The composition may further comprise one or more additional active or inactive agents.

In some embodiments, the subject is diagnosed as having a TBI prior to administration of the PPARγ agonist and/or stem cells or progenitor cells. In some embodiments, the subject is suspected of having a TBI at the time of administration.

TBI may occur when an external force traumatically injures the brain. There are different systems for classifying TBI based on, for example, severity, type of injury and prognosis. The most commonly used system for classifying TBI is the Glasgow Coma Scale (GCS), which grades a person's level of consciousness on a scale of 3-15 based on verbal, motor, and eye-opening reactions to stimuli. In general, a TBI with a GCS score of 13 or above is defined as mild, 9-12 as moderate and 8 or below as severe. Another system, the Mayo Classification System, has three main classifications including definite moderate-severe TBI, probable mild TBI, and possible TBI. Multiple criteria are used in each diagnosis including loss of consciousness, post-traumatic amnesia, skull fracture, and evidence of neuroradiological abnormalities including subdural haematoma, cerebral contusion, and hemorrhagic contusion. The classification of TBI using the GCS or Mayo systems will be known to those skilled in the art.

In some embodiments, a health care provider uses one or more tests that assess a subject's physical injuries, brain and nerve functioning, and level of consciousness. Some of these tests include: gcs; measurements for level of TBI; speech and language tests; cognition and neuropsychological tests; imaging tests (e.g., computerized tomography (CT), magnetic resonance imaging (MM), and intracranial pressure (ICP) monitoring); and tests for assessing TBI in military settings. Biomarkers of TBI may also be detected in a sample from the subject, such as blood, breath, cerebrospinal fluid, or saliva. The biomarker may be any class of biological molecule known to correlate with TBI, likelihood of TBI, or risk of TBI, such as a chemical metabolite, protein, peptide, or micro RNA.

The TBI may be any type of traumatic brain injury, such as concussion, coup injury or contrecoup injury, diffuse axonal injury, or an acquired brain injury such as that which results from damage to the brain caused by stroke, tumor, anoxia, hypoxia, toxin, degenerative disease, near drowning and/or other condition not necessarily caused by an external force. The level of brain injury may be mild (e.g., Glasgow coma scale score of 13-15), moderate (e.g., Glasgow coma scale score of 9-12), or severe (e.g., Glasgow coma scale score of 8 or below).

Common causes of TBI include falls, vehicle-related collisions, violence, sport injuries, and explosive blasts and other combat injuries. TBI also results from penetrating wounds, severe blows to the head with shrapnel or debris, and falls or bodily collisions with objects following a blast.

TBI can cause a wide range of functional short- or long-term changes affecting thinking, sensation, language, or emotions. TBI can also cause epilepsy and increase the risk for conditions such as Alzheimer's disease, Parkinson's disease, and other brain disorders that become more prevalent with age. Repeated mild TBIs occurring over an extended period of time (i.e., months, years) can result in cumulative neurological and cognitive deficits. Repeated mild TBIs occurring within a short period of time (i.e., hours, days, or weeks) can be catastrophic or fatal.

The PPARγ agonist may be an agonist of PPARγ1, or PPARγ2, or both. The PPARγ agonist may be a synthetic compound or natural product. For example, a synthetic PPARγ agonist may be pioglitazone, rosiglitazone, troglitazone, englitazone, balaglitazone, rivoglitazone, ciglitazone, lobeglitazone, or netoglitazone, or a pharmaceutically acceptable salt of any of the foregoing. For example, a natural PPARγ agonist may be honokiol, amorfrutin 1, amorfrutin B, and amorphastilbol, or a pharmaceutically acceptable salt of any of the foregoing.

The PPARγ agonist may be an exogenous agonist, such as a drug, or an endogenous agonist. The PPARγ agonist may be a selective agonist (selective for PPARγ), full agonist (eliciting a maximum response from the PPARγ in one or more tissues), co-agonist, or partial agonist (having partial efficacy at the receptor relative to the full agonist). The PPARγ agonist may be a dual PPAR agonist, such as a PPAR alpha/gamma dual agonist (e.g., LSN862 and saroglitizar). Methods for identifying agents that act as a PPARγ are disclosed in U.S. Patent Publication No. 2004/0235019 (Chapman J et al.), which is incorporated herein by reference in its entirety.

In some embodiments of the compositions and methods of the invention, the one or more PPARγ agonists may be pharmaceutically acceptable salts of compounds, such as pharmaceutically acceptable salts of pioglitazone (e.g., pioglitazone sulfate or pioglitazone hydrochloride; see, for example, U.S. Pat. No. 7,230,016 (Jie Zhu, Frantisek Picha), which is incorporated herein by reference in its entirety).

PPARγ agonists and other compounds used in the invention can be formulated into pharmaceutically-acceptable salt forms. Pharmaceutically-acceptable salts of the compounds of the invention can be prepared using conventional techniques. “Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The stem cells or progenitor cells may be autologous, allogeneic, or xenogeneic to the subject to which they are administered. In some embodiments, the subject is human and the stem cells or progenitor cells are human stem cells or progenitor cells. In some embodiments, the stem cells or progenitor cells are mesenchymal stromal cells or mesenchymal progenitor cells. In some embodiments, the stem cells or progenitor cells are human mesenchymal stromal cells or human mesenchymal progenitor cells. In some embodiments, the stem cells or progenitor cells are neural stem cells or neural progenitor cells.

In some embodiments of the methods and compositions of the invention, the stem or progenitor cells are human mesenchymal stromal cells as described in Horwitz E M et al., Curr Opin Hematol, 2006 November, 13(6):419-425, which is incorporated herein by reference in its entirety.

Methods and markers commonly used to identify stem cells and to characterize differentiated cell types are described in the scientific literature (e.g., Stem Cells: Scientific Progress and Future Research Directions, Appendix E1, E5, report prepared by the National Institutes of Health, June, 2001). The list of adult tissues reported to contain stem cells is growing and includes bone marrow, peripheral blood, umbilical cord blood, brain, spinal cord, dental pulp, blood vessels, skeletal muscle, epithelia of the skin and digestive system, cornea, retina, liver, and pancreas.

The stem cells or progenitor cells are preferably administered in an isolated state. The cells may be genetically modified or non-genetically modified.

Additional agents may be administered to the subject such as immunosuppressive agents and/or additional agents for treatment of the TBI.

PPARγ agonists and other compounds used in the invention may be formulated to enhance solubility, be prepared as prodrugs, or be formulated for controlled or sustained release. Chemical reactions, reactants, and reagents that may be utilized to enhance solubility and make prodrugs of compounds are described in March's Advanced Organic Chemistry, 7^{th} edition, 2013, Michael B. Smith, which is incorporated herein by reference in its entirety.

Compounds (e.g., PPARγ agonists), cells (e.g., stem cells and/or progenitor cells), and compositions comprising them, useful in the methods of the subject invention, can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of at least one compound of the invention is combined with a suitable carrier or diluent in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject peptides and polynucleotides include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of the compounds of the invention can also comprise suspension agents, protectants, lubricants, buffers, preservatives, and stabilizers.

In some embodiments, a PPARγ agonist and progenitor or stem cells are administered in amounts effective to alleviate or eliminate one or more signs and/or symptoms of TBI. In some embodiments, PPARγ agonist and progenitor or stem cells are administered in amounts effective to reduce or eliminate neurodegeneration and/or CCL20 expression in the subject's brain (e.g., cortex), relative to the extent of neurodegeneration and/or CCL20 expression that would occur in the absence of administered PPARγ agonist and progenitor or stem cells. In some embodiments, the reduction of neurodegeneration and/or CCL20 expression is significantly greater than that resulting from the administration of PPARγ agonist or progenitor or stem cells individually.

Examples of physical symptoms of mild TBI include loss of consciousness for a few seconds to a few minutes; a state of being dazed, confused or disoriented, without a loss of consciousness; headache; nausea or vomiting; fatigue or drowsiness; problems with speech; difficulty sleeping; sleeping more than usual; and dizziness or loss of balance. Examples of sensory symptoms of mild TBI include sensory problems, such as blurred vision, ringing in the ears, a bad taste in the mouth or changes in the ability to smell; and sensitivity to light or sound. Examples of cognitive or symptoms of mild TBI include memory or concentration problems; mood changes or mood swings; and feeling depressed or anxious.

Moderate to severe TBI can include any of the signs and symptoms of mild injury, as well as these symptoms that may appear within the first hours to days after a head injury. Examples of physical symptoms of moderate to severe TBI include loss of consciousness from several minutes to hours; persistent headache or headache that worsens; repeated vomiting or nausea; convulsions or seizures; dilation of one or both pupils of the eyes; clear fluids draining from the nose or ears; inability to awaken from sleep; weakness or numbness in fingers and toes; and loss of coordination. Examples of cognitive or mental symptoms of moderate to severe TBI include profound confusion; agitation, combativeness or other unusual behavior; slurred speech; and coma and other disorders of consciousness.

Infants and young children with brain injuries might not be able to communicate headaches, sensory problems, confusion and similar symptoms. In a child with TBI, one may observe: change in eating or nursing habits; unusual or easy irritability; persistent crying and inability to be consoled; change in ability to pay attention; change in sleep habits; seizures; sad or depressed mood; drowsiness; and loss of interest in favorite toys or activities.

PPAR agonists are agents that act upon the peroxisome proliferator-activated receptor. Some are used for the treatment of symptoms of the metabolic syndrome, mainly for lowering triglycerides and blood sugar. PPARγ (gamma) is the main target of the drug class of thiazolidinediones (TZDs), used in diabetes mellitus and other diseases that feature insulin resistance. In some embodiments of the methods and compositions of the invention, the PPARγ agonist is a TZD, such as pioglitazone (PG), or a pharmaceutically acceptable salt thereof. The agonist may be a single or dual agonist (acting on the gamma isoform only, or gamma and another isoform, respectively).

In addition to PG, other examples of PPARγ agonists that may be used include, but are not limited to, Ciglitazone, Edaglitazone, G W 1929, LG 100754, nTZDpa, 15-deoxy-Δ-12,14-Prostaglandin J2, Rosiglitazone, S26948, Telmisartan, and Troglitazone. Other examples can be found in Wang L et al., Biochem Pharmacol. 2014 Nov. 1; 92(1): 73-89; and Swomya P et al., PPAR Research, Volume 2017 (2017), Rocchi S. et al., Molecular Cell, 2001, 8:737-747; Berger J P, et al. Mol Endocrinol, 2003, 17:662-676; Shimaya A, et al., Metabolism, 2000, 49:411-417; Chakrabarti R, et al., Diabetes, 2003, 52 (Suppl. 1) p 601 (Abstract); Kawai T, et al., Metabolism, 1999, 48:1102-1107; and Wulff E, et al., Diabetes, 2003, 52 (Suppl. 1) p 594 (abstract); and U.S. Pat. Nos. 5,089,514; 4,342,771; 4,367,234; 4,340,605; and 5,306,726 which are incorporated herein by reference in their entirety.

Optionally, acetyl L-carnitine, or a pharmaceutically acceptable salt thereof, or another agent may be administered before, during, and/or after the PPAR-γ agonist, in order to alleviate or avoid one or more adverse effects of the PPAR-γ agonist. Such agents may be administered in the same composition as the PPAR-γ agonist or stem cells or progenitor cells, or in a separate composition. The clinical use of the PPAR-γ agonists pioglitazone and rosiglitazone have shown some adverse effects, including weight gain, fluid retention, congestive heart failure, and bone fractures suggesting that these effects are likely PPAR-γ dependent (Nesto R W et al. “Thiazolidinedione use, fluid retention, and congestive heart failure: a consensus statement from the American Heart Association and American Diabetes Association.” Oct. 7, 2003. Circulation, 2003, 108:2941-2948; Betteridge D J. “Thiazolidinediones and fracture risk in patients with type 2 diabetes”, Diabet Med. . 2011; 28:759-771; and Food and Drug Administration. Advisory Committee Briefing Document. Preclinical pharmacology and toxicology summary. Drug: Pargluva® (muraglitazar, BMS-298,585). Bethesda, Md.: Food and Drug Administration; 2005). Acetyl L-carnitine has been reported to prevent or delay the onset of adverse effects of PPAR-γ agonists (U.S. Patent Publication 2010/0305204, Calvani M et al.).

As used herein, the terms “administer”, “apply”, “treat”, and “deliver”, and grammatical variations thereof, are used interchangeably to provide agents such as PPARγ agonists and stem cells or progenitor cells to a subject.

Therapeutic or prophylactic application of the PPARγ agonists and stem or progenitor cells, and the composition or compositions containing them, can be accomplished by any suitable method and technique presently or prospectively known to those skilled in the art for delivery to a subject. Administration of the PPARγ agonists and stem or progenitor cells, and the composition or compositions containing them, can be continuous or at distinct intervals as can be readily determined by a person skilled in the art. For example, cells and PPARγ agonists can be administered to a subject intracranially, intracerebrally, intramuscularly, intradermally, intravascularly (e.g., intravenously), intraocularly, orally, intranasally, topically, or by open surgical procedure, depending upon the anatomical site or sites to which the cells or PPARγ agonists are to be delivered, which may be by different delivery routes. Cells can also be administered in an open manner, or in the brain during stereotactic surgery, or by intravascular interventional methods using catheters, for example.

In some embodiments, administration of the cells and PPARγ agonist to the subject is initiated within 24 hours of the TBI. In some embodiments, administration of the cells and PPARγ agonist to the subject is initiated within 48 hours of the TBI. In some embodiments, administration of the cells and PPARγ agonist to the subject is initiated within 72 hours of the TBI. In some embodiments, administration of the cells and PPARγ agonist to the subject is initiated within 1-2 weeks of the TBI.

In some embodiments, a PPARγ agonist is administered to the subject at intervals (e.g., once a day for two, three, four, or five days) following the TBI, and the stem or progenitor cells are administered after administration of the PPARγ agonist ceases.

A single type of PPARγ agonist may be administered to a subject, or a combination of two or more types of PPARγ agonist may be administered. Progenitor cells may be administered without stem cells, stem cells may be administered without progenitor cells, or a combination or mixture of stem cells and progenitor cells may be administered to the subject.

As used herein, the term “co-administration” and variations thereof refers to the administration of two or more agents simultaneously (in one or more preparations), or consecutively. The PPARγ agonists and stem cells or progenitor cells may be co-administered.

The “PPARγ agonist” used in the invention induce or increase activation or enhance one or more biological activities of the PPARγ, such as reduction of CCL20 expression in the brain (e.g., cortex), decrease in the inflammatory response of many cardiovascular cells, particularly endothelial cells, activation of the PON1 gene, increasing synthesis and release of paraoxonase 1 from the liver, reducing atherosclerosis, degradation of beta-catenin during pre-adipocyte differentiation. Assays and reagents for PPARγ activity and biological effects thereof are known and commercially available. Such assays may be used to screen test samples to quantify functional activity, either agonist or antagonist, that they may exert against human PPARγ. Examples include the PPARγ (human) Reporter Assay Kit, Item No. 15729, and PPARgamma Transcription Factor Assay Kit, Cay-10006855 (Cayman Chemical, Ann Arbor, Mich.); TaqMan assay with Fast Real-Time PCR Universal PCR Master Mix and TaqMan probes (probe ID Hs01011368 ml, Life Technologies, CA, USA); and Primary antibodies for CCL20 (rabbit polyclonal antibody, cat. no. Ab9829, Abcam, Cambridge, UK); and Duo-Set enzyme-linked immunosorbent assay (ELISA) (R&D, Abingdon, UK).

As used herein, the terms “subject”, “patient”, and “individual” refer to a human or non-human animal. Typically, the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human. The subject may be any age or gender. For example, in some embodiments, the subject is elderly, or is a child or adolescent. People most at risk for TBI include children, especially newborns to 4-year-olds; young adults, especially those between ages 15 and 24; adults age 60 and older; and males in any age group.

The term “a,” “an,” “the” and similar terms used in the context of the present invention (especially in the context of the claims) are to be construed to cover both the singular and plural unless otherwise indicated herein or clearly contradicted by the context. For example, the term “cell” includes a singular cell and a plurality of cells unless specified to the contrary; and the term “agonist” or “PPARγ agonist” includes a singular agonist and a plurality of agonists.

### Exemplified Embodiments

Embodiment 1. A method for treatment of traumatic brain injury in a human or non-human animal subject, comprising administering a peroxisome proliferator-activated receptor gamma (PPARγ) agonist, and stem cells or progenitor cells, to the subject.

Embodiment 2. The method of embodiment 1, wherein the PPARγ agonist is administered prior to, during, or after administration of the stem cells or progenitor cells, or any combination thereof.

Embodiment 3. The method of embodiment 1 or 2, wherein the PPARγ agonist comprises two or more PPARγ agonists.

Embodiment 4. The method of any preceding embodiment, wherein the PPARγ agonist is a selective PPARγ agonist.

Embodiment 5. The method of any one of embodiments 1 to 3, wherein the PPARγ agonist is a dual PPAR agonist.

Embodiment 6. The method of any one of embodiments 1 to 3, wherein the PPARγ agonist is selected from among pioglitazone, rosiglitazone, troglitazone, englitazone, balaglitazone, rivoglitazone, ciglitazone, lobeglitazone, or netoglitazone, or a pharmaceutically acceptable salt of any of the foregoing.

Embodiment 7. The method of any one of embodiments 1 to 3, wherein the PPARγ agonist is selected from among honokiol, amorfrutin 1, amorfrutin B, and amorphastilbol, or a pharmaceutically acceptable salt of any of the foregoing.

Embodiment 8. The method of any preceding embodiment, wherein the PPARγ agonist comprises a thiazolidinedione (TZD).

Embodiment 9. The method of embodiment 8, wherein the thiazolidinedione comprises pioglitazone, or a pharmaceutically acceptable salt thereof.

Embodiment 10. The method of any preceding embodiment, wherein the subject is human.

Embodiment 11. The method of any preceding embodiment, wherein the stem cells or progenitor cells are autologous, allogeneic, or xenogenic to the subject.

Embodiment 12. The method of any preceding embodiment, wherein the stem cells or progenitor cells are mesenchymal stromal cells or mesenchymal progenitor cells.

Embodiment 13. The method of any preceding embodiment, wherein the stem cells or progenitor cells are human mesenchymal stromal cells or progenitor cells.

Embodiment 14. The method of any preceding embodiment, wherein the subject is human and the stem cells or progenitor cells are human mesenchymal stromal cells.

Embodiment 15. A pharmaceutical composition comprising stem or progenitor cells, such as mesenchymal stromal cells; and a PPARγ agonist.

Embodiment 16. The pharmaceutical composition of embodiment 11, wherein the PPARγ agonist comprises two or more PPARγ agonists.

Embodiment 17. The pharmaceutical composition of any preceding embodiment, wherein the PPARγ agonist is a selective PPARγ agonist.

Embodiment 18. The pharmaceutical composition of embodiment 15 or 16, wherein the PPARγ agonist is a dual PPAR agonist.

Embodiment 19. The pharmaceutical composition of embodiment 15 or 16, wherein the PPARγ agonist is selected from among pioglitazone, rosiglitazone, troglitazone, englitazone, balaglitazone, rivoglitazone, ciglitazone, lobeglitazone, or netoglitazone, or a pharmaceutically acceptable salt of any of the foregoing.

Embodiment 20. The pharmaceutical composition of embodiment 15 or 16, wherein the PPARγ agonist is selected from among honokiol, amorfrutin 1, amorfrutin B, and amorphastilbol, or a pharmaceutically acceptable salt of any of the foregoing.

Embodiment 21. The pharmaceutical composition of embodiment 15 or 16, wherein the PPARγ agonist comprises a thiazolidinedione (TZD).

Embodiment 22. The pharmaceutical composition of embodiment 15 or 16, wherein the thiazolidinedione comprises pioglitazone, or a pharmaceutically acceptable salt thereof.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Improved Outcome of Stem Cell Therapy for Traumatic Brain Injury Using Lateral Fluid Percussion Injury Model

Adult male SD rats were subjected to lateral fluid percussion injury (LFPI) to induce TBI. Following injury rats were treated with PG or vehicle daily for 3 days and human mesenchymal stromal cells (hMSCs)/rat were administered intranasally on day 3.

Rats were first treated with 2 μg/kg Pioglitazone or vehicle once a day for 3 days post-TBI, and then with or without 10⁶ human mesenchymal stromal cells (hMSCs) intranasally on day 3. 4 days after hMSC treatment rats were euthanized and organs were harvested. IVIS live imaging was performed on organs immediately after harvesting.

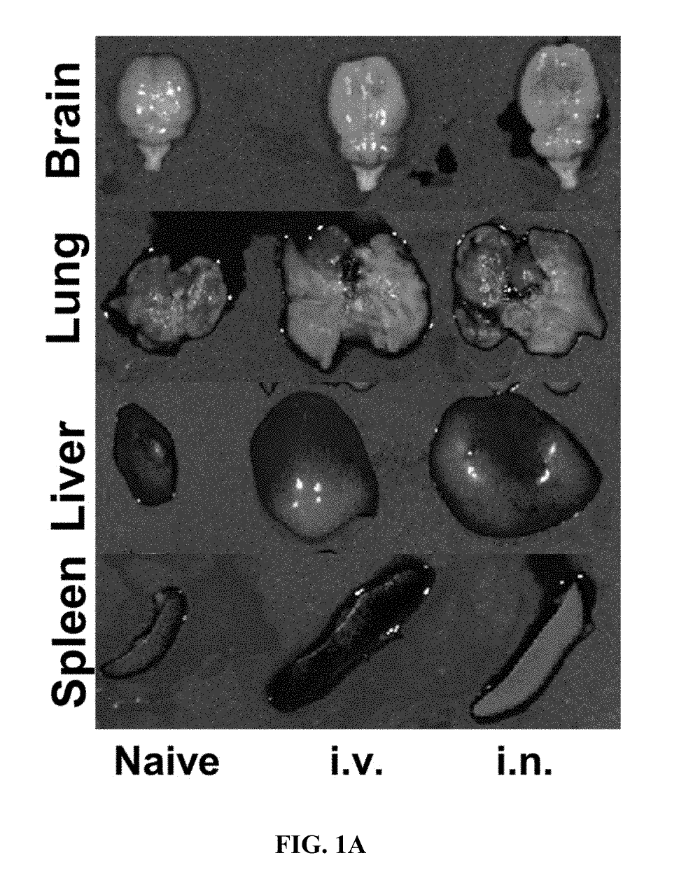

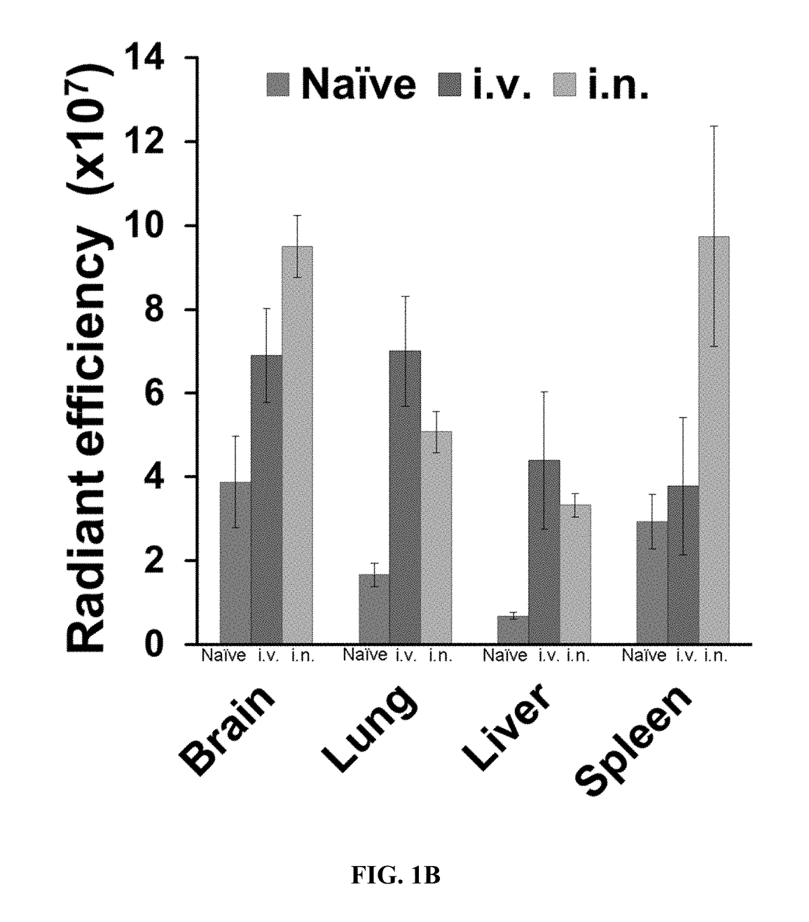

Fluorojade (FJ) and CC120 expressions in the cortex were analyzed in the brain by immunohistochemical method using specific antibodies. Intensity of immunoreactivity was quantitated using image J program. FIGS. 1A-1B show biodistribution and quantitation of hMSCS in rat after TBI.

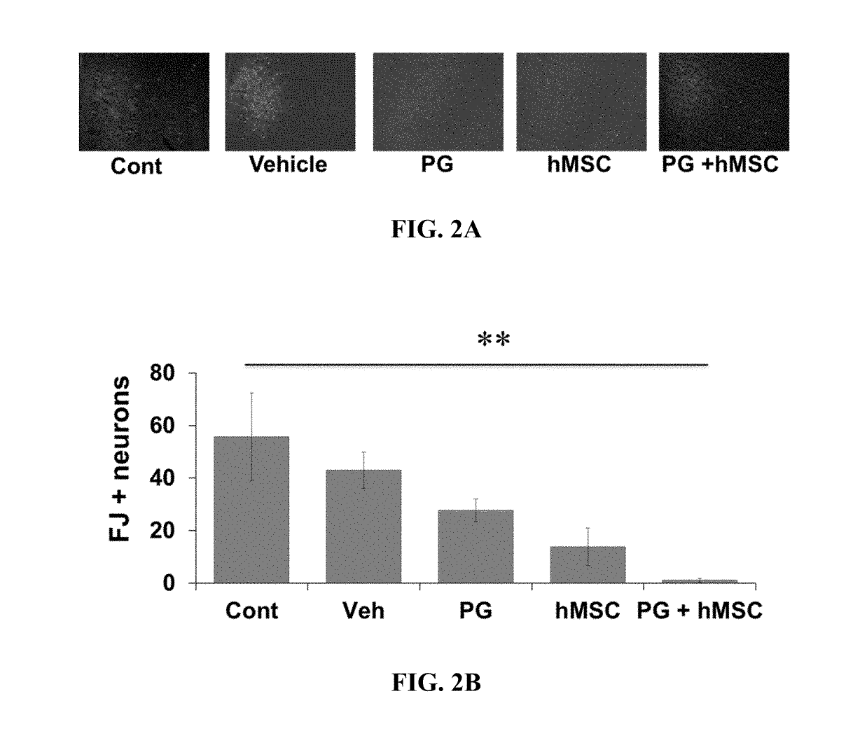

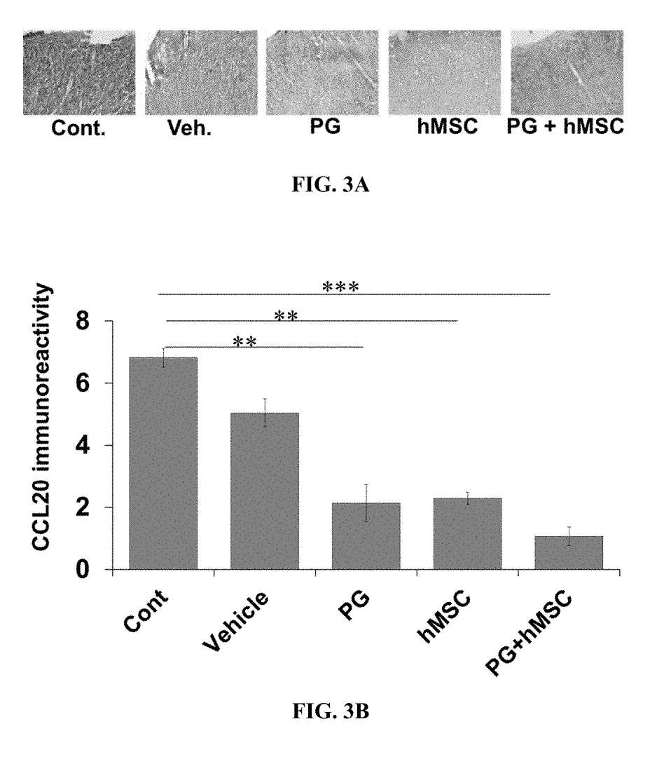

The results showed upregulation of Fluoro-Jade positive, GFAP-positive neurons and CCL20 expression in the cortex 7 days post-TBI. In the PG-treated or hMSC-treated rats, neurodegeneration and CCL20 expression were partially reduced after 7 days post TBI. On the other hand, rats with PG pre-treatment followed by hMSC administration showed a complete reduction of neurodegeneration and CCL20 expression indicating much improved treatment outcome. Results are shown in FIGS. 2A-2B and 3A-3B.

Taken together, the results in these studies demonstrate that improved hMSC treatment outcome following TBI in rats may be achieved by a combination therapy comprising pre-treatment with PG and then hMSC treatment.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Das M, Leonardo C C, Rangooni S, Pennypacker K R, Mohapatra S, Mohapatra S S., J Neuroinflammation, Oct. 31, 2011, 8:148.

Tajiri N, Acosta S A, Shahaduzzaman M D, et al., The Journal of Neuroscience, Jan. 1, 2014, 34(1):313-326.

We claim:

1. A method for treatment of traumatic brain injury in a human or non-human animal subject, comprising administering a peroxisome proliferator-activated receptor gamma (PPARγ) agonist, and stem cells or progenitor cells, to the subject.

2. The method of claim 1, wherein the PPARγ agonist is administered prior to administration of the stem cells or progenitor cells.

3. The method of claim 1, wherein the PPARγ agonist comprises two or more PPARγ agonists.

4. The method of claim 1, wherein the PPARγ agonist is a selective PPARγ agonist.

5. The method of claim 1, wherein the PPARγ agonist is a dual PPAR agonist.

6. The method of claim 1, wherein the PPARγ agonist is selected from among pioglitazone, rosiglitazone, troglitazone, englitazone, balaglitazone, rivoglitazone, ciglitazone, lobeglitazone, or netoglitazone, honokiol, amorfrutin 1, amorfrutin B, and amorphastilbol, or a pharmaceutically acceptable salt of any of the foregoing.

7. The method of claim 1, wherein the PPARγ agonist comprises a thiazolidinedione (TZD).

8. The method of claim 7, wherein the thiazolidinedione comprises pioglitazone, or a pharmaceutically acceptable salt thereof.

9. The method of claim 1, wherein the subject is human.

10. The method of claim 1, wherein the stem cells or progenitor cells are autologous or allogeneic to the subject.

11. The method of claim 1, wherein the stem cells or progenitor cells are mesenchymal stromal cells or mesenchymal progenitor cells.

12. The method of claim 1, wherein the stem cells or progenitor cells are human mesenchymal stromal cells or progenitor cells.

13. The method of claim 1, wherein the subject is human and the stem cells or progenitor cells are human mesenchymal stromal cells.

14. A pharmaceutical composition comprising stem or progenitor cells; and a PPARγ agonist.

15. The pharmaceutical composition of claim 14, wherein the PPARγ agonist comprises two or more PPARγ agonists.

16. The pharmaceutical composition of claim 14, wherein the PPARγ agonist is a selective PPARγ agonist.

17. The pharmaceutical composition of claim 14, wherein the PPARγ agonist is a dual PPAR agonist.

18. The pharmaceutical composition of claim 14, wherein the PPARγ agonist is selected from among pioglitazone, rosiglitazone, troglitazone, englitazone, balaglitazone, rivoglitazone, ciglitazone, lobeglitazone, or netoglitazone, honokiol, amorfrutin 1, amorfrutin B, and amorphastilbol, or a pharmaceutically acceptable salt of any of the foregoing.

19. The pharmaceutical composition of claim 14, wherein the PPARγ agonist comprises a thiazolidinedione (TZD).

20. The pharmaceutical composition of claim 19, wherein the thiazolidinedione comprises pioglitazone, or a pharmaceutically acceptable salt thereof.
